(19) United States
(12) Patent Application Publication
(10) Pub. No.: US 2020/0141838 A1
(43) Pub. Date: May 7, 2020

(54) OFF-LINE BYPASS LOOP ARRANGEMENT FOR A WATER RECYCLING DEVICE

(71) Applicant: ORBITAL SYSTEMS AB, MALMÖ (SE)

(72) Inventor: Michael Ridell, Staffanstorp (SE)

(21) Appl. No.: 16/631,031

(22) PCT Filed: Jul. 4, 2018
(86) PCT No.: PCT/SE2018/050734

§ 371 Date: Jan. 14, 2020

(30) Foreign Application Priority Data
Jul. 14, 2017 (SE) 1750925-8

(51) Int. Cl. G01N 1/20; G01N 33/18; C02F 1/00

(52) U.S. Cl. CPC G01N 1/2035; G01N 33/18; C02F 1/008; G01N 2001/2064; G01N 2001/205; C02F 2209/06; C02F 2209/05; C02F 2209/36; C02F 2209/11

(57) ABSTRACT

The present invention provides a method for measuring one or more parameters in a water flow in a device intended for recycling of water, said method including directing part of the water flow to a liquid-stagnant space where the measuring is performed. The present invention is also directed to a device intended for recycling of water, said device including a flow path for recycled water, a fresh water inlet, a recirculation water inlet and a recirculation water outlet, a user outlet, a heater and a filter, and wherein the device also includes an off-line bypass loop arrangement which includes a liquid-stagnant space and which off-line bypass loop arrangement also includes one or more sensors enabling measuring in the off-line bypass loop arrangement.

### FIELD OF THE INVENTION

The present invention relates to a method for measuring one or more parameters in a water flow in a device intended for recycling of water, to a measuring unit intended for a water recycling device and to a water recycling device comprising such a measuring unit.

### TECHNICAL BACKGROUND

There are many different types of water recirculation systems. One such is shown in WO2013/095278, which describes a hybrid device for a recirculation shower, allowing purification and either recycling of water or discarding of water, wherein said hybrid device comprises a recirculation loop, a filter system with a nano-filter, at least one filter quality sensor, at least one pre-filter, and wherein the hybrid device is arranged to redirect the water from recirculation to drainage when the at least one filter quality sensor indicates the need thereof.

Furthermore, in WO2017/099663 there is also disclosed a water recirculation apparatus, wherein the apparatus comprises a light unit intended for neutralisation of organisms in said water flow, and wherein the light unit, such as a UV LED unit, is arranged in a dockable water treatment chamber.

The present invention also relates to water recirculation systems, e.g. such showers. The present invention is directed to providing an improved measuring method and a measuring unit intended for water recirculation systems.

### SUMMARY OF THE INVENTION

The stated purpose above is achieved by a method for measuring one or more parameters in a water flow in a device intended for recycling of water, said method comprising directing part of the water flow to a liquid-stagnant space where the measuring is performed. The concept of the present invention is to ensure that the measuring is performed on a water amount which is stagnant. This has several advantages, Firstly, as the water amount is not in movement, there is no or a very little risk of air bubbles, which in themselves is an error source for the measurement. Secondly, as the water amount is stagnant, the present invention provides the control of measuring several parameters on one and the same water amount, not only once but also during several times if this is of interest, before a specific water amount is conversed and replaced. Both advantages mentioned above are important when measuring in a system where water is flowing, such as in a water recirculation system, e.g. in a recirculating shower.

The present invention is also directed to a device intended for recycling of water, said device comprising a flow path for recycled water, a fresh water inlet, a recirculation water inlet and a recirculation water outlet, a user outlet, a heater and a filter, and wherein the device also comprises an off-line bypass loop arrangement which comprises a liquid-stagnant space and which off-line bypass loop arrangement also comprises one or more sensors enabling measuring in the off-line bypass loop arrangement. One example of such a device is shown in FIG. 1 and the apparatus concept of the present invention is further described below.

### BRIEF DESCRIPTION OF THE DRAWINGS

FIG. 1 depicts an exemplary device for recycling of water according to an aspect of the present invention.

### DETAILED DESCRIPTION

Below, specific embodiments of the present invention are disclosed. According to one specific embodiment of the present invention, the liquid-stagnant place is arranged as an off-line bypass loop arrangement comprising a quality measuring cell in the device intended for recycling of water. It should be noted that the present invention covers all systems where the liquid is led to liquid-stagnant space for measurement, however an off-line bypass arrangement is one preferred alternative. This off-line bypass arrangement is further discussed below.

According to yet another specific embodiment of the present invention, the step of directing part of the water flow to a liquid-stagnant space is performed cyclically. Cyclically should in this regard be linked to the sampling timing, implying that the method according to the present invention is performed repeatedly. The actual measuring may be performed linked to a minimum off-line regulation, i.e. measuring first after a certain time range when it is ensured that the water flow is stopped. Moreover, the actual measuring frequency for different parameters may vary so that certain parameters, e.g. turbidity, may be measured every sampling cycle, however other parameters, such as microbiological activity, may be measured only once a day or so.

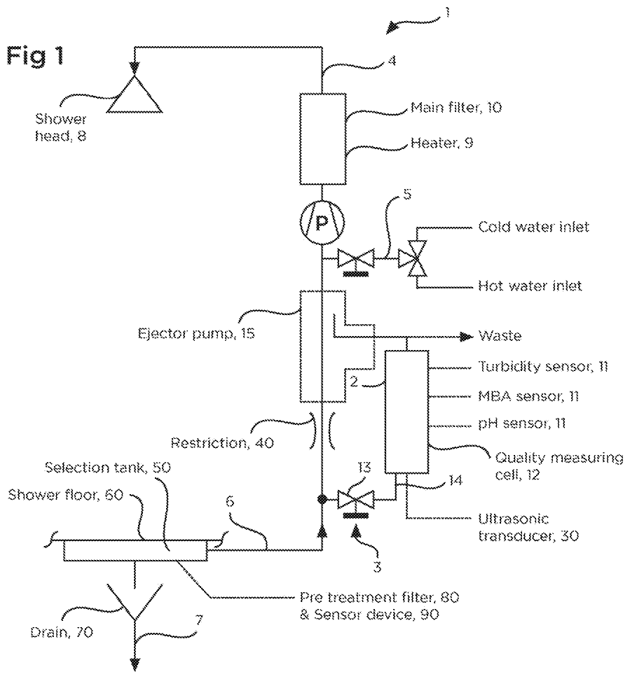

As may be understood from above, the method may involve measuring one or more different parameters. According to one specific embodiment of the present invention, the measuring is performed on one or more of the parameters turbidity, conductivity, pH value, ultrasound and microbiological activity. It should be noted that any combinations of parameters above are possible, and according to one specific embodiment of the present invention, measuring is performed on at least turbidity, which may be a key parameter in several cases. Turbidity is of interest to measure the water quality, and may function as a quality measurement of a primary water quality measurement performed by measuring conductivity (with EC sensor(s)). pH value indicates another important parameter for water. Ultrasound is induced to remove air bubbles, e.g. by use of an ultrasonic transducer (see FIG. 1), but may also be measured by an ultrasound sensor. Measuring the ultrasound may be implemented by measuring the time for ultrasound to travel a known distance, which is also called time of flight (TOF).

Moreover, the present invention may also involve measuring other parameters not mentioned above, such as water hardness. Furthermore, the method according to the present invention may also involve adding a reagent to the liquid-stagnant space before measuring a certain parameter.

According to yet another specific embodiment of the present invention, measuring is performed on turbidity, and reference value is set as a start by measuring penetrated light through fresh water, and this is used as a reference, and wherein the turbidity then is measured by comparing liquid light penetration with reference, and as such enabling to determine water quality. This in itself is different than measuring turbidity according to the standard. According to the present invention, calibration may be performed for a water recycling device and the control system and operation method thereof during the production of the device by using what is known to be fresh water. As such, a reference value may be set, and therefore it may be enough to measure a difference to this reference value, and not an absolute value as is the standard. According to this embodiment of the present invention, the actual measurement is simplified as it is enough to measure the level of light penetrating from one side of a tank or quality measuring cell (see below) or the like containing the liquid-stagnant space.

The present invention also provides a device intended for recycling of water, where said device comprises an off-line bypass loop arrangement which comprises a liquid-stagnant space and which off-line bypass loop arrangement also comprises one or more sensors enabling measuring in the off-line bypass loop arrangement.

According to one specific embodiment of the present invention, the offline bypass loop arrangement comprises a quality measuring cell and a magnetic valve. The actual measuring is being performed in the quality measuring cell and the magnetic valve is provided to ensure a filling of a new measuring amount after emptying the same and when a new measurement is intended.

As is shown in FIG. 1, the entire off-line bypass loop arrangement may comprise a measuring tank which is arranged on a bypass line within the offline bypass loop arrangement, and where the off-line bypass loop arrangement also comprises an ejector pump. The quality measuring cell is emptied by use of the ejector pump. The content therein is either sent up further in the recirculation loop when the water “passes” the quality measurement levels, or otherwise sent to the waste. When exchanging the liquid in the quality measuring cell again, the magnetic valve is opened. Suitably this is performed in the same sequence so that the quality measuring tank is always full, or at least not empty.

According to one embodiment of the present invention, the off-line bypass loop arrangement is arranged as a suction unit on the low pressure side of the device intended for recycling of water. One such alternative is shown in FIG. 1. By using the low pressure side, the utilization sequence is simplified.

The device according to the present invention may comprise one or several sensors. According to one specific embodiment of the present invention, the off-line bypass loop arrangement comprises sensors for measurement of at least one of the parameters turbidity, conductivity, pH value, ultrasound and microbiological activity. According to one specific embodiment, the off-line bypass loop arrangement comprises a turbidity sensor unit.

It should be noted that also other sensors may be arranged, e.g. for measuring the water hardness. The sensors are connected to a control system which is also connected to other sensors or data handling or sourcing units. The data may be used to analyse the incoming water as such, e.g. in the case of water hardness, but the mina purpose is for measuring the real-time water quality in a water recirculating system, such as a recirculating shower. Moreover, the data collected may also give input to decisions being made in the control system, such as with reference to if water should be recirculated or discarded or how often and when cleaning of the device should be performed etc. etc.

As is further discussed below, the off-line bypass loop arrangement may also comprise an ultrasonic transducer. This is further discussed below, also in relation to reducing the error source of air bubbles when using turbidity as a water quality parameter being measured.

In FIG. 1 there is shown a device 1 intended for recycling of water, in this case a recirculating shower, where the device 1 comprises a liquid-stagnant space 2 where measuring is intended to be performed. The liquid-stagnant space 2 is positioned in an off-line bypass loop arrangement 3 of the device 1, which off-line bypass loop arrangement 3 also comprises one or more sensors 1 1 enabling measuring.

Moreover, the device 1 also comprises a flow path 4 for recycled water, a fresh water inlet 5 (cold and hot water inlet), a recirculation water inlet 6 from a selection tank and a recirculation water outlet 7, a user outlet 8 (shower head), a heater 9 and a main filter 10 enabling the recycling feature. As seen in FIG. 1, the water flowed in via the fresh water inlet 5 (cold and hot water inlet) meets a recirculation loop comprising the recirculation water inlet 6, which in fact is one point along the loop before subsequent key units being a heater 9 and filter 10. In the flow path up-streams, the user outlet 8, in this case a shower head, is provided subsequently to the heater 9 and filter 10. As seen in FIG. 1 there is arranged a selection tank 50 being a point from which water is either wasted via the water recirculation outlet (drain 70) or sent to recirculation. As the water recirculation device in this case is a recirculating shower, the selection tank is provided in the shower floor 60. The selection tank 50 suitably comprises a pre-filter or rough filter 80 and a sensor 90. This sensor enables for the system to decide if water should be discarded and sent to the drain or recycled. As seen in the flow path 4 for recycled water, there is provided a heater 9 and a main filter 10, which suitably is arranged in a combined heater and filter unit. Moreover, this unit may also comprise a water treatment source, such as a light unit, e.g. a UV lamp, to be used for killing microorganisms in the water.

Moreover, the off-line bypass loop arrangement 3 comprises a quality measuring tank 12 and a magnetic valve 13. The quality measuring tank 12 may be seen as a conversion tank, and can be a comparatively small unit, such as between 0.5 and 1.5 litres, e.g. between 0.5 and 1.0 litres, in case of recirculation shower as shown in FIG. 1. As seen in FIG. 1, the quality measuring tank 12 is arranged on a bypass line 14 within the off-line bypass loop arrangement 3, and where the off-line bypass loop arrangement 3 also comprises an ejector pump 15. When a new measuring cycled is to be performed the ejector pump 15 sucks water from the mail line as well as the quality measuring tank 12. The restriction 40 is provided to enable the operation of the ejector pump 15. As the magnetic valve 13 is open then also a new water conversion amount is pumped into the measuring tank 15. The measuring is then being performed on this water amount.

Several sensors 11 may be arranged in the off-line bypass loop arrangement 3, suitably in the quality measuring tank 12 as shown in FIG. 1. In this case there is at least provided sensors 1 1 for measuring turbidity, microbiological activity (MBA) and pH value, but other ones may be provided in addition to these on instead of these. Furthermore, an ultrasonic transducer 30 may also be arranged in the quality measuring tank 12 as shown in FIG. 1. This transducer 30 may be used to reduce the number or air bubbles by introducing ultrasound, and may also be used to measure the level of ultrasound being introduced. Air bubbles may affect the measurement of turbidity and therefore it may be of relevance to reduce or diminish the amount of air bubbles.

Moreover, and as shown in FIG. 1, the quality measuring cell 12 is also linked to the waste so that the water amount held therein may be directly sent to the waste if needed.

1. A method for measuring one or more parameters in a water flow in a device intended for recycling of water, said method comprising directing part of the water flow to a liquid-stagnant space where the measuring is performed.

2. The method according to claim 1, wherein the liquid-stagnant place is arranged as an off-line bypass loop arrangement comprising a quality measuring cell in the device intended for recycling of water.

3. The method according to claim 1, wherein the step of directing part of the water flow to a liquid-stagnant space is performed cyclically.

4. The method according to claim 1, wherein the measuring is performed on one or more of the parameters turbidity, conductivity, pH value, ultrasound and microbiological activity.

5. The method according to claim 1, wherein measuring is performed on at least turbidity.

6. The method according to claim 1, wherein measuring is performed on turbidity, wherein a reference value is set as a start by measuring penetrated light through fresh water, and this is used as a reference, and wherein the turbidity then is measured by comparing liquid light penetration with reference, and as such enabling to determine water quality.

7. A device intended for recycling of water, said device comprising a flow path for recycled water, a fresh water inlet, a recirculation water inlet and a recirculation water outlet, a user outlet, a heater and a filter, and wherein the device also comprises an off-line bypass loop arrangement which comprises a liquid-stagnant space and which off-line bypass loop arrangement also comprises one or more sensors enabling measuring in the off-line bypass loop arrangement.

8. The device according to claim 7, wherein the off-line bypass loop arrangement comprises a quality measuring cell and a magnetic valve.

9. The device according to claim 8, wherein the measuring tank is arranged on a bypass line within the off-line bypass loop arrangement, and wherein the off-line bypass loop arrangement also comprises an ejector pump.

10. The device according to claim 7, wherein the off-line bypass loop arrangement is arranged as a suction unit on the low pressure side of the device intended for recycling of water.

11. The device according to claim 7, wherein the off-line bypass loop arrangement comprises sensors for measurement of at least one of the parameters turbidity, conductivity, pH value, ultrasound and microbiological activity.

12. The device according to claim 7, wherein the off-line bypass loop arrangement comprises an ultrasonic transducer.

13. The device according to claim 7, wherein the off-line bypass loop arrangement comprises a turbidity sensor unit.
